(19) United States
(12) Patent Application Publication
(10) Pub. No.: US 2024/0226417 A1
(43) Pub. Date: Jul. 11, 2024

(54) CODED HOUSING COMPONENTS FOR AN INJECTION DEVICE

(71) Applicant: Sanofi, PARIS (FR)

(72) Inventors: Beate Franke, Frankfurt am Main (DE); Michael Helmer, Frankfurt am Main (DE)

(21) Appl. No.: 18/289,298

(22) PCT Filed: May 2, 2022
(86) PCT No.: PCT/EP2022/061643

§ 371 Date: Nov. 2, 2023

(30) Foreign Application Priority Data
May 3, 2021 (EP) 21315073.3

(51) Int. Cl. A61M 5/00

(52) U.S. Cl. CPC A61M 5/002

(57) ABSTRACT

A coded housing of a drug delivery device includes a first housing component having a first connecting end a second housing component having a second connecting end, an insert on the first connecting end or the second connecting end, a receptacle on the other of the first connecting end or the second connecting end, a fastening element on the insert, a counter fastening element complementary shaped to the fastening element and provided in the receptacle, a mechanical coding on the insert and comprising a coding feature, and a mechanical counter coding in the receptacle. The mechanical coding and the mechanical counter coding is operable to prevent an engagement of the fastening element with the counter fastening element when the mechanical coding does not match the mechanical counter coding.

### CROSS REFERENCE TO RELATED APPLICATIONS

The present application is the national stage entry of International Patent Application No. PCT/EP2022/061643, filed on May 2, 2022, and claims priority to Application No. EP 21315073.3, filed on May 3, 2021, the disclosures of which are incorporated herein by reference.

### TECHNICAL FIELD

The present disclosure relates to the field of drug delivery devices and systems, particularly to injection devices for injecting a liquid medicament.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easily understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump. With respect to reusable injection or delivery devices, a patient may have to load or to replace a cartridge. Reusable injection devices typically comprise a multi-component housing. For instance, the housing may comprise a proximal housing component, such as a body and a distal housing component, such as a cartridge holder detachably connectable to the body. Once a medicament provided in a medicament container, such as a cartridge, is empty, the cartridge holder may be disconnected from the body of the injection device and the empty cartridge may be removed and replaced with a new cartridge.

Another concern may arise from cartridges being manufactured in essentially standard sizes and manufactured to comply with certain recognized local and international standards. Consequently, such cartridges are typically supplied in standard sized cartridges (e.g. 3 ml cartridges). Therefore, there may be a variety of cartridges supplied by a number of different suppliers and containing a different medicament but fitting a single drug delivery device. As just one example, a first cartridge containing a first medicament from a first supplier may fit a drug delivery device provided by a second supplier. As such, a user might be able to load an incorrect medicament into a drug delivery device and, then, dispense said medicament (such as a rapid or basal type of insulin) without being aware that the medical delivery device was perhaps neither designed nor intended to be used with such a cartridge.

### SUMMARY

The present disclosure is directed to drug delivery devices and systems comprising a multi-component housing, wherein one housing component is configured to accommodate a medicament container, such as a cartridge and wherein another housing component is configured to accommodate a drive mechanism to operably engage with the medicament container for expelling or withdrawing a dose of the medicament.

There is a growing desire from users, health care providers, caregivers, regulatory entities, and medical device suppliers to reduce the potential risk of a user loading an incorrect drug type into a drug delivery device. It is also desirable to reduce the risk of dispensing an incorrect medicament (or the wrong concentration of the medicament) from such a drug delivery device. There is a general need to physically dedicate or mechanically code a cartridge and/or cartridge holder to its drug type and design an injection device that only accepts or works with the dedication or coded features provided on or with the cartridge and/or cartridge holder so as to prevent unwanted cartridge cross use. Similarly, there is also a general need for a dedicated cartridge that allows the medical delivery device to be used with only an authorized cartridge containing a specific medicament while also preventing undesired cartridge cross use.

In one aspect, the disclosure relates to a housing of a drug delivery device, in particular to a housing of an injection device, such as a handheld injection pen. The housing comprises a first housing component configured to accommodate a cartridge filled with a medicament. The first housing component comprises a first connecting end. The housing further comprises a second housing component. The second housing component is configured to accommodate a drive mechanism of the drug delivery device. Typically, the drive mechanism comprises a piston rod extending in longitudinal direction and configured to operably engage with a piston or bung of the cartridge for expelling a dose of the medicament from the cartridge.

The second housing component comprises a second connecting end. Typically, the first connecting end is connectable to the second connecting end to form or to constitute the housing of the drug delivery device. With some examples the first housing component is an elongated or tubular shaped housing component comprising the first connecting end at a longitudinal proximal end. The second housing component may be also of tubular or elongated shape. The second connecting end may be located at a distal longitudinal end of the second housing component.

There is further provided an insert on one of the first connecting end and the second connecting end. The insert is typically integrally formed with the respective first or second housing component. There is further provided a receptacle on the other one of the first connecting end and the second connecting end. The insert is insertable into the receptacle along the longitudinal direction for mutually fastening the first housing component and the second housing component and/or for forming or establishing the housing of the drug delivery device. Typically, the receptacle is provided at one of the first and second connecting ends and forms a respective connecting end. The insert is provided on the other one of the first and second connecting ends and forms a respective connecting end.

The receptacle comprises an inner cross-section sized and shaped to receive the insert therein. Typically, an inside diameter or inside cross-section of the receptacle closely matches an outside diameter or outer cross-section of the insert.

The housing further comprises a fastening element provided on the insert and a counter fastening element complementary shaped to the fastening element and provided in the receptacle. Typically, and when reaching a final assembly configuration the fastening element engages the counter fastening element thereby fastening and fixing the first housing component to the second housing component; and vice versa.

The housing further comprises a mechanical coding provided on the insert. The mechanical coding comprises a coding feature. The housing further comprises a mechanical counter coding provided in the receptacle and comprising a counter coding feature. The mechanical coding and the mechanical counter coding are operable to prevent an engagement of the fastening element with the counter fastening element when the mechanical coding does not match the mechanical counter coding. A mutual assembly of the first housing component and the second housing component and/or a mutual engagement of the fastening element and the counter fastening element requires that a first housing component provided with a mechanical coding is assembled with a second housing component provided with a corresponding or complementary-shaped mechanical counter coding.

The mechanical coding is defined by one of a lateral extent in the cross-sectional geometry or shape of the coding feature in a plane transverse to the longitudinal direction. The mechanical counter coding is defined by one of a lateral extent and a cross-sectional geometry or shape of the counter coding feature in the plane transverse to the longitudinal direction. With some examples the coding feature is represented by a diameter, in particular an outer diameter of the insert. The counter coding feature is represented by a diameter, in particular an inner diameter of the receptacle. With some examples, the insert is of tubular shape and the receptacle is of a complementary tubular shape, wherein the inside diameter of the receptacle is substantially equal to the outside diameter of the insert.

With other examples the insert comprises a well-defined or dedicated cross-sectional geometry. It may comprise one of an oval, triangular, quadratic or polygonal outer shape that matches a complementary oval, triangular, quadratic or polygonal inner shape of the receptacle.

Only when the mechanical coding of the insert matches the mechanical counter coding in the receptacle the fastening element and the counter fastening element will be enabled to mutually engage. With all other pairings or combinations of a mechanical coding, e.g. of a first type, with a non-matching mechanical counter coding, e.g. of a second type, the fastening element and the counter fastening element are hindered from mutually engaging. Then, a mutual assembly and/or a fixing of first and second housing components is effectively prevented.

Mutual engagement of the fastening element with the counter fastening element requires that the mechanical coding matches the mechanical counter coding. In this way, unintended cross use of a first housing component of a first drug delivery device with a second housing component of another drug delivery device can be effectively prevented.

Prevention of the mutual engagement of the fastening element with the counter fastening element can be effectively achieved in two different ways. According to some examples the mechanical coding and the non-matching mechanical counter coding are configured to prevent insertion or a complete insertion of the insert into the receptacle along the longitudinal direction. Here, and with a coding on the insert non-matching with the counter coding of the receptacle the insert may be mechanically blocked from entering the receptacle. Alternatively, the insert may be sized and shaped to enter the receptacle but then the coding non-matching with the counter coding is configured to prevent an engagement of the fastening element with the counter fastening element. In effect, for a coding non-matching with a counter coding the first housing component cannot be connected or fixed to the second housing component.

Implementation of a coding that is defined by a lateral extent of an insert of the first housing component is of particular advantage when the first housing component is to be equipped with cartridges of different size, in particular with cartridges of different diameter. The first housing component with a mechanical coding of a first type may distinguish from a first housing component with a mechanical coding of a second time by its lateral extent, in particular by its diameter. In this way, the first housing component provided with the mechanical coding of a first type may comprise a diameter that is smaller than the diameter of a first housing component provided with a mechanical coding of the second type. Varying of the diameter of a sidewall of the first housing component, in particular of a sidewall portion of the insert of the first housing component provides a rather failure safe and robust mechanical coding effectively preventing an unintended cross use of a first housing component with a mechanical coding of a first type to with a second housing component provided with a non-matching counter coding of a second or third type.

In general, references to a lateral extent of the one of the insert and the receptacle include a respective reference to the cross-sectional geometry, the cross-sectional shape or cross-sectional diameter of the insert and/or of the receptacle; and vice versa.

According to a further example the mechanical coding is integrated into a sidewall portion of the insert. The insert may be of tubular shape. The complementary shaped receptacle may be also of tubular shape. Integrating the mechanical coding into the sidewall portion of the insert may be provided by varying the diameter, in particular by varying the outer diameter of the insert. The mechanical coding feature may be represented by the outer diameter of the insert. Different and mutually distinguishing coding features of the mechanical coding can be provided by varying the lateral extent of the insert, at least by varying the diameter, in particular by varying the outer diameter of the insert of the first housing component.

According to a further example the mechanical coding is defined by an outer diameter, an outer cross-section or an outer shape of the insert. In other words, the coding feature of the mechanical coding is the outer diameter, the outer cross-section or the outside shape of the insert.

Varying of the lateral extent of the coding feature and hence of the insert may disable insertion of a cartridge of non-matching or inappropriate size into the respective housing component. In this way a cartridge having a diameter being larger than the inner diameter of the insert cannot be inserted in longitudinal direction into or beyond the insert of the respective housing component. In this way, e.g. when the insert is provided on the first housing component, a variation of the diameter of the insert may prevent inserting of a cartridge having a diameter that is larger than a cartridge dedicated for use with this particular first housing component.

According to a further example the mechanical counter coding is integrated into a side wall of the receptacle. The sidewall of the receptacle may be of tubular shape. It may be designed and configured for receiving the insert in longitudinal direction. Integrating the mechanical counter coding into the sidewall of the receptacle, in particular integrating the mechanical counter coding into the inside of the sidewall of the receptacle provides a robust and unequivocal mechanical counter coding such that only an insert of appropriate size can be inserted in longitudinal direction into the receptacle. Integration of the mechanical counter coding on the inside of the sidewall of the receptacle may be invisible from outside the housing. In this way the mechanical counter coding can be concealed when the first and the second housing components are mutually assembled and/or interconnected.

In this way a kit of numerous housings of a drug delivery device that distinguish by their mechanical codings and counter codings may exhibit the same or identical outer appearance when the first and the second housing components are mutually assembled.

According to a further example the mechanical counter coding is defined by an inside diameter, an inside cross-section or by an inside shape of the receptacle. The diameter, the cross-section or inside shape of the receptacle refer to the plane transverse to the longitudinal direction. When the mechanical coding does not match the mechanical counter coding the insert is either hindered to enter the receptacle, to reach a final assembly position inside the receptacle or when reaching a final position of assembly, the transverse extent or shape of the insert may be smaller than the inside extent or inside dimensions of the receptacle such that the fastening element provided on the insert cannot engage the complementary-shaped counter fastening element provided in the receptacle.

Here, and when reaching a final assembly position with regard to the longitudinal direction the fastening element may be separated laterally or radially from the counter fastening element. The fastening element and the counter fastening element may be located at a radial or lateral distance, thereby inherently preventing a mutual engagement of the fastening element and the counter fastening element. Establishing of a mechanical connection between the first housing component and the second housing component is thus effectively prevented and/or inhibited.

According to a further example and when the mechanical coding does not match the mechanical counter coding an outer diameter, an outer cross-section or an outside shape of the insert is larger than a respective inside diameter, inside cross-sectional shape or geometry of the receptacle. In this way an insert motion of the insert into the receptacle in longitudinal direction is effectively blocked and prevented.

According to a further example the mechanical coding is integrated into the fastening element and the mechanical counter coding is integrated into the counter fastening element. Here, the fastening element and the counter fastening element may contribute to the lateral extent of the insert and to the lateral extent of the receptacle, respectively. Here, and with some examples an outside diameter or outer lateral extent of the sidewall of the insert may even match the inside diameter or inner lateral or radial extent of the receptacle. The fastening element and the counter fastening element may comprise a pair of a radial protrusion and a radial recess. The radial protrusion and the radial recess of the fastening element and the counter fastening element contributes to the lateral extent of the insert and of the receptacle, respectively.

Insofar, the fastening element and the counter fastening element contribute to the lateral extent of the insert and of the receptacle, respectively. With some examples and even when the lateral extent of the sidewall of the insert matches the lateral extent of the sidewall of the receptacle the coding and the counter coding may be entirely or at least partially defined by the lateral extent of a radial protrusion of one of the fastening element and the counter fastening element. Here, a radial protrusion of at least one of the fastening element and the counter fastening element may be either too large or too small to engage with a complementary shaped radial recess of the other one of the fastening element and the counter fastening element.

In this way the mechanical coding and the complementary-shaped mechanical counter coding may be entirely defined by the radial extent or radial size of a pair of at least a fastening element provided on the insert and a complementary-shaped counter fastening element provided in the receptacle.

According to another example one of the fastening element and the counter fastening element comprises a radial recess to engage with a complementary shaped radial protrusion of the other one of the fastening element and the counter fastening element. When the mechanical coding or the mechanical coding feature is integrated into the fastening element and when the mechanical counter coding is integrated into the counter fastening element the mechanical coding feature and mechanical counter coding features may be defined by the lateral or radial extent of the radial protrusion and the radial recess, respectively.

With some examples the coding may be entirely defined by the radial or lateral extent of the fastening element and/or the respective counter coding may be entirely defined by the radial extent or lateral extent of the counter fastening element while the lateral or radial extent of the sidewall of the insert and/or the lateral or radial extent of the sidewall of the receptacle remain invariant for different types of codings and counter codings.

With other examples the size and shape of the fastening element and counter fastening element remains invariant for different types of codings and counter codings while the lateral or radial extent of the sidewall of the insert and the sidewall of the receptacle is subject to geometric variations to provide a unique mechanical coding and counter coding, respectively.

According to a further example the fastening element comprises a snap element to engage with a complementary-shaped counter snap element of the counter fastening element. The snap element and the counter snap element may be engaged by a movement relative to each other along the longitudinal direction. At least one of the snap element and the counter snap element comprises a radial protrusion while the other one of the snap element and the counter snap element comprises a complementary or correspondingly shaped radial recess. Typically, at least one of the protrusion and the recess is elastically deformable in radial or transverse direction. In this way and when arriving in a final position of assembly the snap element and the counter snap element mutually engage due to an elastic relaxation in radial or transverse direction such that the protrusion engages the complementary-shaped recess. By implementing the fastening element and the counter fastening element as a snap connection there will be provided a haptic as well as an acoustic feedback for the user when the first and second housing component reach a final assembly configuration, in which the snap element and the counter snap element mutually engage.

Typically, there are provided numerous snap elements and complementary-shaped counter snap elements on the insert and in the receptacle, respectively. For instance, there may be provided numerous snap elements along the outer circumference of the insert. There may be provided a corresponding number of complementary-shaped counter snap elements on the inside circumference of the insert. In this way, multiple snap fit engagements between the fastening element and the counter fastening element can be provided. This provides increased stability and rigidity of the mechanical connection between the first housing component and the second housing component.

According to a further example and when the mechanical coding does not match the mechanical counter coding an outer diameter, an outer cross-section or an outside shape of the insert is smaller than a respective inside diameter, inside cross-section or inside shape of the receptacle less a radial extent of the radial protrusion of one of the fastening element and the counter fastening element. In other words, the difference of the lateral extent of the outside of the insert and the inside of the receptacle is larger than or at least equal to the radial extent of the radial protrusion of one of the fastening element and the counter fastening element. In this way it can be effectively prevented that the fastening element engages the counter fastening element when the insert reaches a final assembly position inside the receptacle.

Here, the radial distance between the fastening feature and the counter fastening feature is larger than a radial extent of the protrusion of one of the fastening feature and the counter fastening feature. In this way the radial protrusion of one of the fastening element cannot engage the radial recess of the other one of the fastening element and the counter fastening element. A mutual fixing or connection of first and second housing component is effectively prevented even when the insert is insertable into the receptacle.

According to a further example the housing a groove that is provided on one of the insert and the receptacle. The groove extends along the longitudinal direction. Typically, the groove is linearly or straight shaped and extends exclusively in the longitudinal direction or parallel to the longitudinal direction. The housing further comprises a projection provided on the other one of the insert and the receptacle. The projection is configured, hence, the projection is sized and/or shaped to slide along the groove upon insertion of the insert into the receptacle. Mutual engagement of the groove and the projection rotationally locks the first housing component relative to the second housing component during the mutual assembly of the first and second housing components.

The groove has a particular depth in radial or transverse direction. The depth of the groove substantially matches the radial or transverse extent of the projection. When the projection is provided in the receptacle it protrudes radially inwardly. When projection is provided on the insert it protrudes radially outwardly. With either implementation the groove is complementary shaped to the projection.

Typically, the groove adjoins a longitudinal end face of the insert or of the sidewall of the receptacle. Likewise, the projection may adjoin a longitudinal end face of one of the insert and the sidewall of the receptacle. In this way the groove and the projection require or define a mutual orientation of the first housing component relative to the second housing component with regards to an axis of rotation extending parallel to the longitudinal direction. Insertion of the insert into the receptacle is then only possible when the first housing component is in a dedicated orientation relative to the second housing component. During insertion of the insert into the receptacle the first housing component is rotationally locked to the second housing component through the projection sliding along the groove.

According to a further example the fastening element comprises a snap element. Here, the mechanical coding is defined by at least one of a longitudinal position and a longitudinal extent of the snap element on the insert. With this example the fastening element is integrally formed with the mechanical coding. The mechanical coding and the fastening element may coincide. In other words, the fastening element may provide a double or twofold function. It may provide mutual fastening or fixing of first and second housing components. Additionally, it may prevent a pairing or a mutual assembly of non-matching first and second housing components.

According to another example the mechanical counter coding is defined by at least one of a longitudinal position and a longitudinal extent of the counter snap element in the receptacle. Like the fastening element also be counter fastening element is integrally formed with the mechanical counter coding. The mechanical counter coding and the counter fastening element may coincide.

According to a further example the receptacle comprises another counter fastening element located diametrically opposite to the counter fastening element. With further examples, the sidewall of the receptacle is elastically deformable to increase a radial distance between the counter fastening element and the another counter fastening element to such an extent that is larger than or equal a radial distance between the fastening element and another fastening element, which is located diametrically opposite on the insert. In this way and by providing an elastically deformable sidewall of the receptacle the radial distance between diametrically oppositely located counter fastening elements can be increased to such an extent or degree that they disengage from corresponding or complementary shaped fastening elements provided on the insert.

Accordingly, the fastening elements may releasably engage with correspondingly or complementary shaped counter fastening elements. The first and second housing components can be disconnected on demand.

In order to enable or to provide a sufficient elastic deformability of the sidewall of the receptacle it is of particular benefit, when the sidewall of the receptacle at least in the region of its insert opening comprises a comparatively thin side wall. Typically, the sidewall and hence the receptacle and/or the entirety of the respective housing component is made of a plastic material, e.g. an injection molded plastic material inherently providing a sufficient degree of elasticity.

According to a further example the sidewall of the receptacle comprises an outside surface. The outside surface comprises a first flat section and a second flat section radially opposite, e.g. diametrically opposite to the first flat section. A first imaginary straight line intersecting the first flat section and the second flat section extends substantially perpendicular to a second imaginary straight line intersecting the fastening element and the another fastening element.

The first and second flat sections are configured as engaging sections for a particular squeezing tool by way of which the radial distance between the first and second flat sections can be decreased to a predefined degree. A decrease of the radial distance between the first and second flat sections inherently leads to an increase of the radial distance between the first fastening element and the another fastening element. Hence, by applying a radially inwardly directed pressure in opposite directions onto the first and second flat sections the receptacle may adapt or conform a somewhat oval shape, wherein the long axis of the oval shape coincides with the second imaginary straight line intersecting the fastening element and the another fastening element.

The flat section may not only provide a well-defined gripping and squeezing of the housing component but may also provide a visual and haptic guidance for using the squeezing tool.

According to a further example the first housing component or the second housing component comprises a sidewall. The sidewall comprises a first longitudinal section and a second longitudinal section adjoining the first longitudinal section. The first and second longitudinal sections do not overlap. The first longitudinal section may be regarded as a longitudinal extension of the second longitudinal direction; and vice versa. The first and second longitudinal sections may be integrally formed. The insert is formed by the second longitudinal section. A lateral extent, a cross-sectional geometry or cross-sectional shape of the insert and hence of the second longitudinal section distinguishes from a respective lateral extent, cross-sectional geometry or shape of the first longitudinal section of the sidewall.

With some examples the inside diameter of the second longitudinal section distinguishes from the inside diameter of the first longitudinal section. The first and the second longitudinal section may comprise a tubular shape. They may comprise a tubular shape on the inside. They may also comprise a tubular shape on the outside. With some examples the inside diameter of the second longitudinal section differs from the inside diameter of the first longitudinal section. With some examples the inside diameter of the second longitudinal section is smaller than the inside diameter of the first longitudinal section. With other examples the inside diameter of the second longitudinal section is larger than the inside diameter of the first longitudinal section.

With some examples the outside diameter of the second longitudinal section is smaller than the outside diameter of the first longitudinal section. With other examples the outside diameter of the second longitudinal section is larger than the outside diameter of the first longitudinal section.

With some examples the coding or counter coding is entirely provided by varying the lateral extent of the second longitudinal section by leaving the lateral extent or lateral dimensions of the first longitudinal section of the sidewall unchanged. In this way only the insert of the respective housing component is subject to variations in order to provide a mechanical coding while the first longitudinal section of the sidewall of the respective first or second housing component remains unchanged. Here, the mechanical coding can be effectively concealed when the housing is fully assembled, i.e. when the insert is received inside the receptacle.

A variation of the lateral extent of the second longitudinal section of the first or second housing component and hence of the insert provides a rather robust and unequivocal mechanical coding for the respective housing component.

According to a further example the first longitudinal section adjoins the second longitudinal section the via a radial step. When the second longitudinal section of the sidewall comprises a diameter that is larger than a diameter of the first longitudinal section the second longitudinal section is stepped radially outwardly relative to the first longitudinal section. The radial step may be provided on the outside surface and/or on the inside surface of the respective sidewall of that one of the first or second housing components that comprises the insert. The radial step provides a unique and easily distinguishable mechanical coding feature. The radial step size may define the coding feature. The size or extent of the radial or lateral step may be larger than the radial or lateral extent of a protrusion of the fastening element or counter fastening element.

According to another aspect, there is provided an injection device for injecting a dose of a medicament. The injection device comprises a housing as described above and a cartridge arranged inside the housing. The cartridge comprises a barrel filled with a medicament and sealed in a proximal longitudinal direction by a movable bung. The injection device further comprises a drive mechanism arranged inside the housing. The drive mechanism comprises a piston rod operable to exert a distally directed dispensing force onto the bung of the cartridge. Typically, the injection device is implemented as a hand held or portable injection device. The injection device may comprise a pen-type injector.

With some examples the receptacle is provided as a housing insert fixedly attachable or fixedly attached to an elongated housing component, e.g. the first or second housing component of the housing of the drug delivery device. The housing insert may be rotationally and/or longitudinally fixed to the elongated housing component. Insofar all features and benefits as described above in connection with the receptacle equally apply to a housing insert fixedly connectable or fixedly connected to a respective housing component.

According to another aspect, the disclosure relates to a kit of at least a first housing as described above and a second housing as described above. The coding feature of the first housing distinguishes from the coding feature of the second housing with regard to at least one of a lateral extent, a cross-sectional geometry or with regard to a cross-sectional shape of the insert in a plane transverse to the longitudinal direction.

Likewise, the counter coding feature of the first housing distinguishes from the counter coding feature of the second housing with regard to at least one of a lateral extent, a cross-sectional geometry, or a cross-sectional shape of the receptacle in the plane transverse to the longitudinal direction.

The first housing is provided with a pair of a coding and a counter coding of a first type. The second housing is provided with a pair of a coding and a counter coding of a second type. The coding of the first type is unable to pair or to engage the counter coding of the second type. Vice versa, the counter coding of the first type is unable to pair or to engage with the coding of the second type.

Only the coding of the first type is able and configured to pair or to engage with the counter coding of the first type. The coding of the second type is only and exclusively engageable or connectable to the counter coding of the second type; and vice versa.

According to a further example at least one of an outer lateral extent and an outer cross-sectional geometry or outer shape of the insert of the first housing in the plane transverse to the longitudinal direction is larger than a respective inner lateral extent, inner cross-sectional geometry or inner shape of the receptacle of the second housing.

Alternatively, the outer lateral extent, the outer cross-sectional geometry or outer shape of the insert of the first housing in the plane transverse to the longitudinal direction is smaller than a respective inner lateral extent, inner cross-sectional geometry or inner shape of the receptacle of the second housing less a radial extent of a radial protrusion of one of the fastening element or counter fastening element.

When there are provided numerous radial protrusions, e.g. located diametrically oppositely on the outside surface of the insert or on the inside surface of the receptacle, the outer lateral extent of the insert is smaller than the inner lateral extent of the receptacle less twice the radial extent of the respective radial protrusions or less the sum of the radial extent of the respective radial protrusions.

In this way a coding non-matching with a counter coding may be obtained by reducing the diameter of the insert relative to the inner diameter of the insert to such an extent that the fastening element provided on the outside surface of the insert is unable to engage with a complementary-shaped counter fastening element provided on the inside surface of the receptacle.

Generally, and with some examples the first housing components of different housings may distinguish by the size and/or geometry of an accommodating space for receiving a medicament container or cartridge. In particular, a housing with a coding of a first type may be exclusively equipped with a first cartridge or medicament container. A housing with a coding of a second type may be exclusively equipped with a cartridge or a second medicament container. For this, medicament containers, cartridges as well as the interior of the first housing components may comprise further codings or coding features or may distinguish with regard to their size or geometry such that only one dedicated cartridge or medicament container unequivocally fits into only one dedicated first housing component.

With some examples, the first housing component is provided with a mechanical coding to engage with a complementary shaped counter coding of a cartridge. With further examples the first housing component may be provided with at least one of an electronic, a visual or optical coding configured to match with a complementary counter coding of the cartridge, which is also of electronic, visual or optical type.

Moreover, at least one of the cartridge and the first housing component may be provided with a locking or fastening feature by way of which a cartridge can be fixed and/or retained in the first housing component. Here, the first housing component, e.g. implemented as a cartridge holder, and a cartridge assembled therein can be provided as a pre-fabricated housing assembly or as a dedicated cartridge-cartridge holder combination.

In either way, it can be assured or provided that a particular medicament provided in a particular cartridge is unequivocally associated with a particular type of a first housing component, i.e. with a particularly mechanically encoded first housing component. In effect and with some examples, a cartridge provided with a particular medicament can be only accommodated in a correspondingly shaped first housing component equipped with a respective mechanical coding.

With further examples a pre-fabricated housing assembly or a dedicated cartridge-cartridge holder combination is commercially distributed by a pharmaceutical manufacturer. Here, the cartridge may be undetachably or irremovably fixed inside the first housing component and the pharmaceutical manufacturer provides a respective matching between a cartridge filled with a particular medicament and a suitable first housing component, which is mechanically encoded in accordance to the type of medicament located inside the cartridge.

According to a further aspect, the present disclosure also relates to a kit of injection devices. The kit of injection devices comprises at least a first injection device comprising a first housing provided with a coding and a counter coding both of a first type and further comprises a second injection device with a second housing provided and equipped with a coding and a counter coding both of a second type non-matching with the respective counter coding or coding of the first type.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (SIRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, numerous examples of injection devices with dedicated or coded housing components will be described in greater detail by making reference to the drawings, in which:

FIG. 1 schematically illustrates an example of a drug delivery device,

FIG. 2 shows an example of an exploded view of the drug delivery device of FIG. 1,

FIG. 3 shows an example of first and second housing components of the injection device when mutually assembled,

FIG. 4 shows an example of first and second housing components with a coding and a counter coding of a first type before assembly,

FIG. 5 shows an example of first and second housing component with a coding and a counter coding of a second type before assembly,

FIG. 6 shows an enlarged cross-section through the connecting ends of the first and second housing components with a coding matching a counter coding,

FIG. 7 shows an enlarged cross-section through the connecting ends of the first and second housing components with a coding non-matching a counter coding,

FIG. 8 shows a perspective view of an example of a first and a second housing component before assembly, and

FIG. 9 shows a perspective view of another example of a first and a second housing component before assembly.

### DETAILED DESCRIPTION

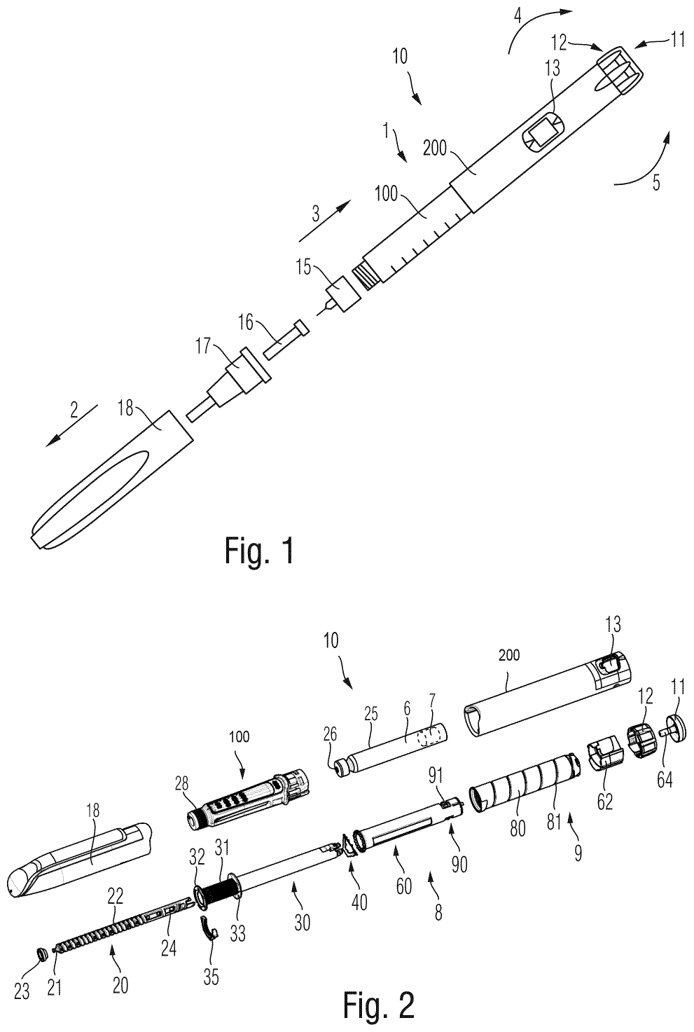

In FIGS. 1 and 2 only one of numerous examples of a handheld injection device is illustrated, that is generally usable in combination with a wearable electronic device. The device as shown in FIGS. 1 and 2 is a pre-filled disposable injection device that comprises a housing 10 to which an injection needle 15 can be affixed. The injection needle 15 is protected by an inner needle cap 16 and either an outer needle cap 17 or a protective cap 18 that is configured to enclose and to protect a distal section of the housing 10 of the injection device 1. The housing 10 comprises a first housing component 100 and a second housing component 200. The second housing component may form a main housing part configured to accommodate a drive mechanism 8 and/or a dose setting mechanism 9 as shown in FIG. 2. The first housing component 100 is configured as a cartridge holder. It may be permanently or releasably connected to the second housing component 200.

The first housing component 100 is typically configured to accommodate a cartridge 6 that is filled with a liquid medicament. The cartridge 6 comprises a cylindrically-shaped or tubular-shaped barrel 25 sealed in proximal direction 3 by means of a bung 7 located inside the barrel 25. The bung 7 is displaceable relative to the barrel 25 of the cartridge 6 in a distal direction 2 by means of a piston rod 20. A distal end of the cartridge 6 is sealed by a pierceable seal 26 configured as a septum and being pierceable by a proximally directed tipped end of the injection needle 15. The cartridge holder and hence the first housing component 100 comprises a threaded socket 28 at its distal end to threadedly engage with a correspondingly threaded portion of the injection needle 15. By attaching the injection needle 15 to the distal end of the first housing component 100 the seal 26 of the cartridge 6 is penetrated thereby establishing a fluid transferring access to the interior of the cartridge 6.

When the injection device 1 is configured to administer e.g. human insulin, the dosage set by a dose dial 12 at a proximal end of the injection device 1 may be displayed in so-called international units (IU, wherein 1 IU is the biological equivalent of about 45.5 μg of pure crystalline insulin (1/22 mg). The dose dial 12 may comprise or may form a dose dial.

As shown further in FIGS. 1 and 2, the housing 10, e.g. the second housing component 200 comprises a dosage window 13 that may be in the form of an aperture in the housing 10. The dosage window 13 permits a user to view a limited portion of a number sleeve 80 that is configured to move when the dose dial 12 is turned, to provide a visual indication of a currently set dose. The dose dial 12 is rotated on a helical path with respect to the housing 10 when turned during setting and/or dispensing or expelling of a dose.

The injection device 1 may be configured so that turning the dosage dial 12 (e.g., dosage knob) causes a mechanical click sound to provide acoustical feedback to a user. The click sound is typically generated by a click noise generator 45. Generally, a click noise generator 45 may be implemented in various different ways. The number sleeve 80 mechanically interacts with a piston in the insulin cartridge 6. When the needle 15 is stuck into a skin portion of a patient, and when the trigger 11 or injection button is pushed, the dose displayed in dosage window 13 (e.g., display window) will be ejected from injection device 1. When the needle 15 of the injection device 1 remains for a certain time in the skin portion after the trigger 11 is pushed, the dose is actually injected into the patient's body. Ejection of a dose of the liquid medicament may also cause a mechanical click sound, which is however different from the click sound produced when using the dose dial 12. For this, the injection device one may comprise a separate, hence a second click noise generator (not illustrated).

In this embodiment, during delivery of the insulin dose, the dose dial 12 is turned to its initial position in an axial movement, that is to say without rotation, while the number sleeve 80 is rotated to return to its initial position, e.g. to display a dose of zero units.

The injection device 1 may be used for several injection processes until either the cartridge 6 is empty or the expiration date of the medicament in the injection device 1 (e.g. 28 days after the first use) is reached.

An example of the drive mechanism 8 is illustrated in more detail in FIG. 2. It comprises numerous mechanically interacting components. A flange like support of the housing 10 comprises a threaded axial through opening threadedly engaged with a first thread or distal thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod 20 rotates relative to the housing 10 thereby experiencing a distally directed advancing motion relative to the housing 10 and hence relative to the barrel 25 of the cartridge 6. As a consequence, the bung 7 of the cartridge 6 is displaced in distal direction 2 by a well-defined distance due to the threaded engagement of the piston rod 20 with the housing 10.

The piston rod 20 is further provided with a second thread 24 at its proximal end. The distal thread 22 and the proximal thread 24 are oppositely handed.

There is further provided a drive sleeve 30 having a hollow interior to receive the piston rod 20. The drive sleeve 30 comprises an inner thread threadedly engaged with the proximal thread 24 of the piston rod 20. Moreover, the drive sleeve 30 comprises an outer threaded section 31 at its distal end. The threaded section 31 is axially confined between a distal flange portion 32 and another flange portion 33 located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further comprises a radial recess or protrusion at its outer circumference to engage with a complementary-shaped recess or protrusion at an inside of the sidewall of the housing 10. In this way the last dose limiter 35 is splined to the housing 10, e.g. to the second housing component 200. A rotation of the drive sleeve 30 in a dose incrementing direction 4 or clockwise direction during consecutive dose setting procedures leads to an accumulative axial displacement of the last dose limiter 35 relative to the drive sleeve 30. There is further provided an annular spring 40 that is in axial abutment with a proximally facing surface of the flange portion 33. Moreover, there is provided a tubular-shaped clutch 60. At a first end the clutch 60 is provided with a series of circumferentially directed saw teeth. Towards a second opposite end of the clutch 60 there is located a radially inwardly directed flange.

Furthermore, there is provided a dose dial sleeve also denoted as number sleeve 80. The number sleeve 80 is provided outside of the spring 40 and the clutch 60 and is located radially inward of the housing 10. A helical groove 81 is provided about an outer surface of the number sleeve 80. The housing 10 is provided with the dosage window 13 through which a part of the outer surface of the number sleeve 80 can be seen. The housing 10 is further provided with a helical rib at an inside sidewall portion of an insert piece 62, which helical rib is to be seated in the helical groove 81 of the number sleeve 80. The tubular shaped insert piece 62 is inserted into the proximal end of the housing 10. It is rotationally and axially fixed to the housing 10. There are provided first and second stops on the housing 10 to limit a dose setting procedure during which the number sleeve 80 is rotated in a helical motion relative to the housing 10.

The dose dial 12 in form of a dose dial grip is disposed about an outer surface of the proximal end of the number sleeve 80. An outer diameter of the dose dial 12 typically corresponds to and matches with the outer diameter of the housing 10. The dose dial 12 is secured to the number sleeve 80 to prevent relative movement there between. The dose dial 12 is provided with a central opening.

The trigger 11, also denoted as dose button is substantially T-shaped. It is provided at a proximal end of the injection device 1. A stem 64 of the trigger 11 extends through the opening in the dose dial 12, through an inner diameter of extensions of the drive sleeve 30 and into a receiving recess at the proximal end of the piston rod 20. The stem 64 is retained for limited axial movement in the drive sleeve 30 and against rotation with respect thereto. A head of the trigger 11 is generally circular. The trigger side wall or skirt extends from a periphery of the head and is further adapted to be seated in a proximally accessible annular recess of the dose dial 12.

To dial a dose a user rotates the dose dial 12. With the spring 40, also acting as a click noise generator 45, and the clutch 60 engaged, the drive sleeve 30, the spring 40, the clutch 60 and the number sleeve 80 rotate with the dose dial 12. Audible and tactile feedback of the dose being dialed is provided by the spring 40 and by the clutch 60. Torque is transmitted through saw teeth between the spring 40 and the clutch 60. The helical groove 81 on the number sleeve 80 and a helical groove in the drive sleeve 30 have the same lead. This allows the number sleeve 80 to extend from the housing 10 and the drive sleeve 30 to climb the piston rod 20 at the same rate. At a limit of travel a radial stop on the number sleeve 80 engages either with a first stop or a second stop provided on the housing 10 to prevent further movement in a first sense of rotation, e.g. in a dose incrementing direction 4. Rotation of the piston rod 20 is prevented due to the opposing directions of the overall and driven threads on the piston rod 20.

The last dose limiter 35 keyed to the housing 10 is advanced along the threaded section 31 by the rotation of the drive sleeve 30. When a final dose dispensed position is reached, a radial stop formed on a surface of the last dose limiter 35 abuts a radial stop on the flange portion 33 of the drive sleeve 30, preventing both, the last dose limiter 35 and the drive sleeve 30 from rotating further.

Should a user inadvertently dial beyond the desired dosage, the injection device 1, configured as a pen-injector allows the dosage to be dialed down without dispense of the medicament from the cartridge 6. For this the dose dial 12 is simply counter-rotated. This causes the system to act in reverse. A flexible arm of the spring 40 or clicker then acts as a ratchet preventing the spring 40 from rotating. The torque transmitted through the clutch 60 causes the saw teeth to ride over one another to create the clicks corresponding to dialed dose reduction. Typically, the saw teeth are so disposed that a circumferential extent of each saw tooth corresponds to a unit dose. Here, the clutch may serve as a ratchet mechanism.

As an alternative or in addition the ratchet mechanism 90 may comprise at least one ratchet feature 91, such as a flexible arm on the sidewall of the tubular-shaped clutch 60. The at least one ratchet feature 91 may comprise a radially outwardly extending protrusion e.g. on a free end of the flexible arm. The protrusion is configured to engage with a correspondingly shaped counter ratchet structure on an inside of the number sleeve 80. The inside of the number sleeve 80 may comprise longitudinally shaped grooves or protrusions featuring a saw-tooth profile. During dialing or setting of a dose the ratchet mechanism 90 allows and supports a rotation of the number sleeve 80 relative to the clutch 60 along a second sense of rotation 5, which rotation is accompanied by a regular clicking of the flexible arm of the clutch 60. An angular momentum applied to the number sleeve 80 along the first sense of rotation for is unalterably transferred to the clutch 60. Here, the mutually corresponding ratchet features of the ratchet mechanism 90 provide a torque transmission from the number sleeve 80 to the clutch 60.

When the desired dose has been dialed the user may simply dispense the set dose by depressing the trigger 11. This displaces the clutch 60 axially with respect to the number sleeve 80 causing dog teeth thereof to disengage. However, the clutch 60 remains keyed in rotation to the drive sleeve 30. The number sleeve 80 and the dose dial 12 are now free to rotate in accordance with the helical groove 81.

The axial movement deforms the flexible arm of the spring 40 to ensure the saw teeth cannot be overhauled during dispense. This prevents the drive sleeve 30 from rotating with respect to the housing 10 though it is still free to move axially with respect thereto. The deformation is subsequently used to urge the spring 40 and the clutch 60 back along the drive sleeve 30 to restore the connection between the clutch 60 and the number sleeve 80 when the distally directed dispensing pressure is removed from the trigger 11.

The longitudinal axial movement of the drive sleeve 30 causes the piston rod 20 to rotate through the through opening of the support of the housing 10, thereby to advance the bung 7 in the cartridge 6. Once the dialed dose has been dispensed, the number sleeve 80 is prevented from further rotation by contact of at least one stop extending from the dose dial 12 with at least one corresponding stop of the housing 10. A zero dose position may be determined by the abutment of one of axially extending edges or stops of the number sleeve 80 with at least one or several corresponding stops of the housing 10.

The expelling mechanism or drive mechanism 8 as described above is only exemplary for one of a plurality of differently configured drive mechanisms that are generally implementable in a disposable pen-injector. The drive mechanism as described above is explained in more detail e.g. in WO2004/078239A1, WO 2004/078240A1 or WO 2004/078241A1 the entirety of which being incorporated herein by reference.

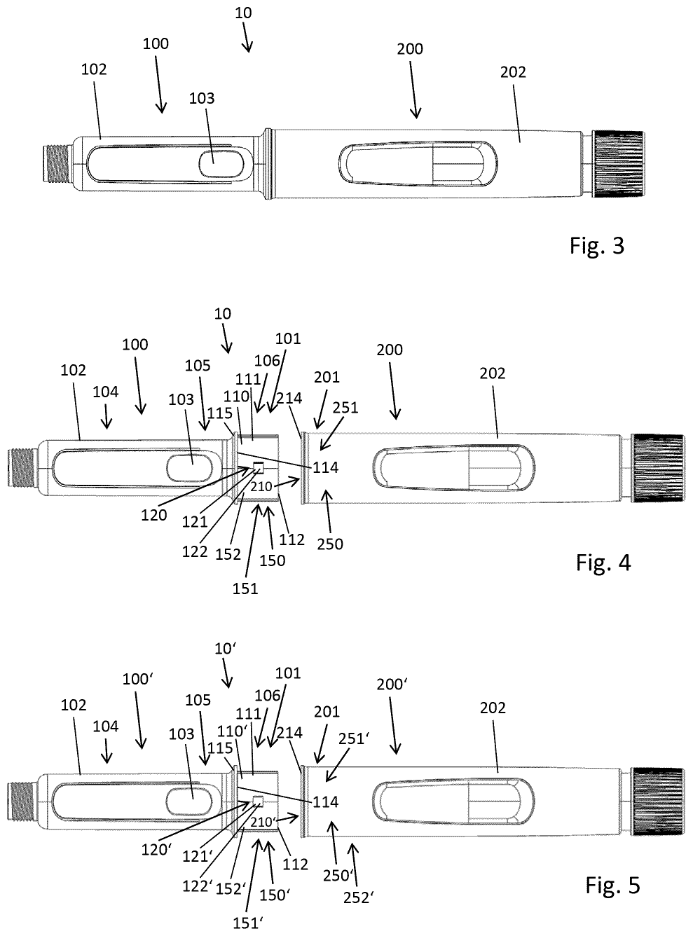

The housing 10 as illustrated in the numerous examples of FIGS. 3-9 comprises a first housing component 100 and a second housing component 200. The first housing component 100 is configured as a cartridge holder. It is sized and shaped to accommodate a cartridge 6 inside its hollow interior. The cartridge holder and hence the first housing component 100 comprises a first connecting end 101. The first connecting end 101 forms a proximal end of the first housing component 100. Correspondingly, the second housing components 200 comprises a second connecting end 201, typically at a distal end of the housing component 200.

The first connecting end 101 is mechanically connectable to the second connecting end 201. As illustrated, the first housing component 100 comprises an insert 110 forming the first connecting end 101. The second housing component 200 comprises a receptacle 210 shaped and sized to receive the insert 110. The insert 110 is insertable into the receptacle 210 by a longitudinal sliding movement relative to the second housing component 200, in particular along the proximal direction 3.

The insert 110 forms a proximal end of the first housing component 100. The insert 110 comprises a proximal end face 112. Towards the distal direction 2 the insert 110 is confined by a flange section 115 protruding radially outwardly from the tubular shaped sidewall 102 of the first housing component 100 and hence also from a sidewall 102 of insert 110.

The flange section 115 comprises a circumferential rim extending all around the tubular shaped insert 110. Towards the proximal direction 3 the flange section 115 comprises an abutment face 114 facing in proximal direction 3. The abutment faces 114 is configured to axially abut a distal end face 214 of a sidewall 202 of the second housing component 200.

For mutually fixing the first and second housing components 100, 200 there is provided a fastening element 120 on a sidewall portion 111 of the insert 110 to operably engage with a correspondingly or complementary-shaped counter fastening element 220 provided inside the receptacle 210. The counter fastening element 220 is provided on an inside surface 203 of the sidewall 202 confining the receptacle 210. In the presently illustrated examples, as for instance shown in greater detail in the cross section of FIG. 6 the fastening element 120 comprises a snap element 121 configured to engage with the correspondingly or complementary shaped counter snap element 221 as provided on an inside surface 203 of the sidewall 202 of the receptacle 210. The snap element 121 comprises a radial recess 122 shaped and configured to engage with and/or to receive a complementary shaped radial protrusion 222 of the counter fastening element 220 protruding radially inwardly from the sidewall 202 of the receptacle 210.

As shown in FIG. 6, there are provided numerous fastening elements 120 and complementary-shaped counter fastening elements 220 on the outside surface of the sidewall 102 of the insert 110 and on the inside surface 203 of the sidewall 202 of the receptacle 210, respectively. By way of mutually corresponding snap elements 121 and counter snap elements 221 a snap-fit engagement of the first and second housing components 100, 200 can be provided when a final assembly configuration of the first and second housing components 100, 200 has been reached, i.e. when the abutment face 114 of the flange section 115 axially engages or abuts the distal end face 214 of the sidewall 202.

There is further provided a mechanical coding 150 on the insert 110. The mechanical coding 150 is complementary shaped to a mechanical counter coding 250 as provided in the receptacle 210. The mechanical coding 150 comprises a mechanical coding feature 151. The mechanical coding 150 is defined by one of a lateral extent and a cross-sectional geometry or shape of the coding feature 151 in a plane transverse to the longitudinal direction (z). In other words, the mechanical coding 150 may be defined by the lateral or radial extent of the insert 110. Insofar, the insert 110 may represent or constitute a coding portion 152 of the first housing component. Likewise, the mechanical counter coding 250 is defined by the radial or lateral extent of the receptacle 210. Accordingly, the counter coding feature 251 and the respective counter coding portion 252 can be represented by or may coincide with the inner geometry or inside extent of the receptacle 210.

An example of a coding 150 matching with a complementary-shaped counter coding 250 and hence an example of a coding and counter coding of a first type is schematically illustrated in FIG. 6. Here, the outside diameter of the insert 110 closely matches an inside diameter of the receptacle 210. Moreover, the longitudinal position of the fastening element 120 on the insert 110 relative to the abutment face 114 matches the longitudinal position of the complementary shaped counter fastening element 220 relative to the distal end face 214.

With a matching pair of a coding 150 and a counter coding 250 the insert 110 is smoothly insertable into the receptacle 210. When reaching a final assembly position or final assembly configuration as illustrated in FIG. 6 the snap element 121 of the fastening element 120 engages the counter snap element 221 of the counter fastening element. In other words, the radial protrusion 222 engages the complementary shaped radial recess 122.

With the example of a coding 150 of a first type as illustrated in FIG. 6 the sidewall 102 of the first housing component 100 comprises a first longitudinal section 104 and a second longitudinal section 106. The second longitudinal section 106 longitudinally adjoins the first longitudinal section 104. The second longitudinal section 106 is of substantially tubular shape and extends proximally from the flange section 115. The first longitudinal section 104 is also of substantially tubular shape and extends in distal direction 2 from the flange section 115. The first longitudinal section 104 and the second longitudinal section 106 merge in the region of the flange section 115 or by the flange section 115.

The second longitudinal section 106 may be configured and implemented as a longitudinal extension of the first longitudinal section 104. Vice versa, the first longitudinal section 104 may be configured and implemented as a distally extending longitudinal extension of the second longitudinal section 106. As further illustrated in FIG. 6 the sidewall 102 and hence the first longitudinal section 104 may be provided with a window 103. The window 103 may be implemented as a through recess extending through the sidewall 102 of the first housing component 100.

With the mechanical coding 150 as illustrated in FIG. 6 the second longitudinal section 106 comprises a lateral extent or a radial diameter that is larger than a respective radial extent or diameter of the first longitudinal section. There is provided a radial step in a transition zone between the first longitudinal section 104 and the second longitudinal section 106. Here, the second longitudinal section 106 is stepped radially outwardly compared to the first longitudinal section 104. An inner diameter D1 of the first longitudinal section 104 is smaller than an inner diameter D2 of the second longitudinal section 106. Accordingly, there will be an annular gap 116 between an inside surface of the second longitudinal section 106 and an outside surface of the barrel 25 of the cartridge 6.

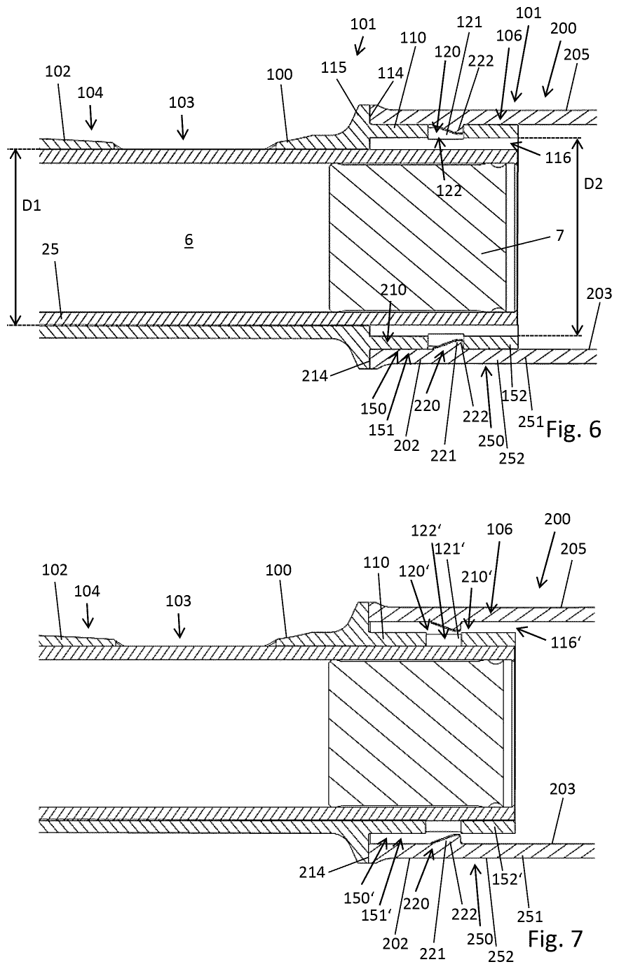

In FIG. 4, a first housing 10 of a kit of multiple housings is illustrated. In FIG. 5, a second housing 10′ of the kit of multiple housings is illustrated. The first housing 10 comprises a first housing component 100 provided with a first mechanical coding 150, i.e. a mechanical coding 150 of a first type. The first housing 10 further comprises a second housing component 200 provided with a first mechanical counter coding 250, a mechanical counter coding 250 of a first type. The mechanical coding 150 and the complementary-shaped mechanical counter coding 250 form a coded pair of a first type.

The second housing 10′ comprises a first housing component 100′ provided with a first mechanical coding 150′, i.e. a mechanical coding of a second type. The second housing 10′ further comprises a second housing component 200′ provided with a second mechanical counter coding 250′, i.e. a mechanical counter coding of a second type.

The mechanical coding 150′ comprises a mechanical coding feature 151′ and forms or constitutes a coding portion 152′. The mechanical coding feature 151's provided by the diameter or lateral extent of the insert 110′. Correspondingly, the mechanical counter coding feature 251′ is provided by the diameter or the lateral extent of the inside of the receptacle 210′.

Compared to the mechanical coding 150 as illustrated in FIG. 6 the mechanical coding 150′ as illustrated in FIG. 7, representing a coding 150′ of a second type distinguishes from the coding 150 of the first type as illustrated in FIG. 6 by the lateral or radial extent of the second longitudinal section 106. There, in FIG. 6 the diameter or lateral extent of the second longitudinal section 106 is substantially equal to the lateral extent or diameter of the first longitudinal section 104. The lateral or radial thickness of the sidewall 102 and of the longitudinal sections 104, 106 of the sidewall 102 may be substantially equal or constant.

With other examples the thickness of the sidewall, e.g. the thickness of the sidewall in the region of the second longitudinal section 106 may be subject to variations in accordance to the respective coding of a first, second or third type.

With the example of FIG. 7 the mechanical coding 150′ of a second type is inserted into a receptacle 210 provided with a mechanical counter coding 250 of a first type.

The radial size or extent of the mechanical coding 150′, the mechanical coding feature 151′ and of the respective coding portion 152′ of the second type is smaller than the respective extent of the coding 150, the coding feature 151 and the coding portion 152 of the first type to such an extent that the outside surface of the insert 110′ is located at a radial distance from an inside surface 203 of the receptacle 210 by a radial gap 116′. The radial gap 116′ comprises a radial extent that is larger than or is at least equal to the radial extent of the radial protrusion 222. In this way, and when reaching a final assembly configuration as illustrated in FIG. 7 the radial protrusion 222 of the counter snap element 221 of the counter fastening element 220 provided or associated with a mechanical counter coding 250 of a first type cannot engage the radial recess 122′ of the snap feature 121′ of the fastening element 120′ provided or associated with a mechanical coding 150′ of a second type. A mutual fixing of the first and second housing components 100, 200 is effectively prevented.

The size of the gap 116′ is equal to or slightly larger than a radial extent of the radial protrusion 222. If there are provided several protrusions 222, e.g. arranged around the inner circumference of the receptacle 210, the outer diameter of the insert 110 is smaller than the inner diameter of the receptacle 210 at least by the sum of the radial extent of at least two oppositely located radial protrusions 222. The mechanical coding 150′ of the second type is dedicated and configured for insertion into a receptacle 210′ provided with a mechanical counter coding 250′ of the second type as indicated in FIG. 5.

The mechanical counter coding 250 of the second type comprises a respective counter coding feature 251′ with a respective coding portion 252′. The counter coding feature 251′ and the counter coding portion 252′ are defined by the inside extent or inside diameter of the receptacle 210′. The receptacle 210′ and hence the counter coding feature 251′ of the second type matches the outside dimensions or outside radial or lateral extent of the insert 110′ of the second type Accordingly, the insert 110 with the coding 150 of the first type cannot be inserted into the receptacle 210′ provided with the counter coding 250′ of the second type. The outer diameter of the insert 110 with the coding feature 151 of the first type is larger than an inside diameter of the receptacle 210′ and hence larger than the radial or lateral extent of the counter coding feature 251′ of the second type. Accordingly, the insert 110 with the mechanical coding 150 of the first type cannot be inserted in longitudinal direction into the receptacle 210′ provided with the counter coding 250′ of the second type.

When comparing the coding 150 of the first type with the coding 150′ of the second type as illustrated in FIGS. 6 and 7, it is apparent that only the second longitudinal section 106 of the sidewall 102 is subject to a diameter variation while the first longitudinal section 104, e.g. located distally from the flange section 115, remains unamended. Here, and for different types of codings or counter codings there may be provided an invariant first longitudinal section 104.

The same or the like may apply to the receptacle 210, 210′ of the counter coding 250 of the first type and the counter coding 250′ of the second type. There, it may be only the inside diameter of the receptacle 210 that varies from the inside diameter of the receptacle 210′ while the outside diameter or outside surface 205 of the second housing component provided with different counter codings 250, 250′ remains unchanged or invariant. In this way, and when the first and second housing components 100, 200 are mutually assembled, the coding 150 matching the counter coding 250 is no longer visible. It is effectively concealed.

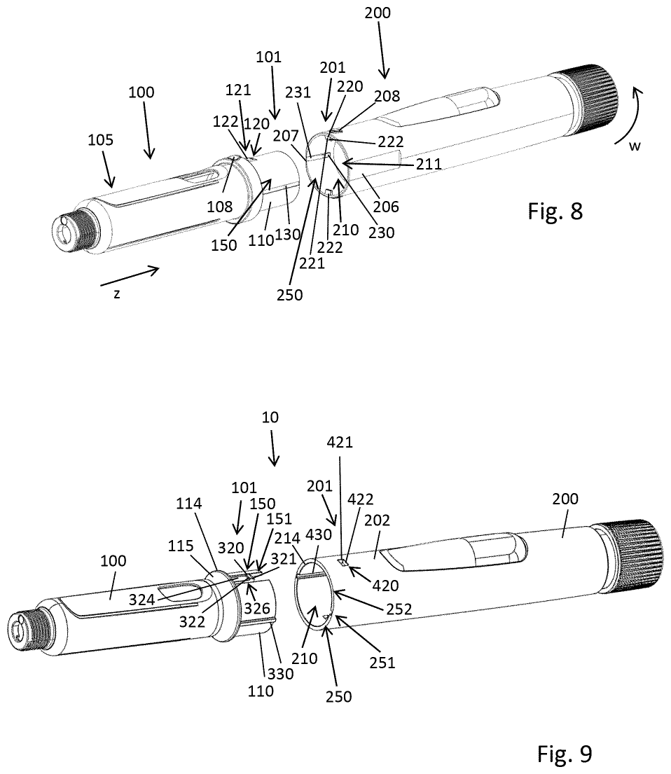

With the example of FIG. 8 there is further illustrated a longitudinal groove 130 on the outside surface of the insert 110. The longitudinal groove 130 extends from the proximal end face 112 of the insert 110 to the flange section 115. Typically, there are provided two diametrically oppositely located longitudinal grooves 130. On the inside surface 203 of the receptacle 210 there is provided a complementary shaped projection 230 shaped and sized to engage with and to slide along the groove 130 upon insertion of the insert 110 into the receptacle 210. The projection 230 and the groove 130 provide a keyed engagement of the insert 110 and the receptacle 210. By way of the keyed engagement the insert 110 and the first housing component 100 is rotationally locked to the receptacle 210 and to the housing components 200. Thus, a mutual fixing and fastening of the first housing component 100 and the second housing component 200 is exclusively obtained by a non-rotational longitudinal sliding motion of the insert 110 into the receptacle 210.

As further illustrated in FIGS. 6 and 7 there is provided an indicator 108 on the outside surface 105 of the first housing component 100. There is provided a complementary or correspondingly shaped indicator 208 on the outside surface 205 of the second housing components 200. When reaching a final assembly configuration, the indicator 108 and the indicator 208 are mutually aligned in longitudinal direction (z). This way, and in the course of inserting the insert 110 into the receptacle 210 the indicator 108 and the indicator 208 provide a visual guiding for the user how to align or to orient the insert 110 relative to the receptacle 210 to support a smooth and longitudinally directed sliding insertion of the insert 110 into the receptacle 210.

With the example of FIG. 8 the projection 230 further comprises a chamfered end section 231 towards the distal end and towards the insert opening 211. In this way, the radial size of the projection 230 reduces towards the distal direction. This provides a rather smooth engagement of the projection 230 with the elongated groove 130 on the insert 110. Moreover, the size reduction of the projection 230 towards the insert opening 211 facilitates and supports an elastic deformation of the sidewall 202 in the region of the insert opening 211, especially when the insert 110 is located inside the receptacle 210. This may support and facilitate an elastic deformation of the sidewall 202.

The flat sections 206, 207 are located on the outside surface 205 of the sidewall 202, e.g. in the region of or longitudinally adjacent to the insert opening 211, and hence at or near a distal connecting end 201 of the second housing component 200.

The first and second flat sections 206, 207 provide a well-defined engagement with a squeezing tool, such as pliers (not illustrated). By applying a radially inwardly directed pressure onto the oppositely located flat sections 206, 207, the radial distance or cross-section between the flat sections 206, 207 can be reduced, thereby increasing the radial distance between the counter fastening elements 220 located opposite to each other on the inside surface 203 of the sidewall 202. Accordingly, the original and somewhat circular cross-section of the insert opening 211 is elastically deformed to adapt a somewhat oval shape.

By increasing the radial distance between the counter fastening elements 220, the counter fastening element(s) 220 may disengage from the complementary shaped fastening element(s) 120 as provided on the insert 110. In this way, the first and second housing components 100, 200 may disengage and can be disassembled. Disengagement of first and second housing component 100, 200 may allow replacement of an empty cartridge 6 and a further use of the drug delivery device 1 with a new cartridge 6.

With the further example of FIG. 9, the implementation of the projection 230 with the complementary shaped groove 130 is somewhat comparable to FIG. 8. With FIG. 9, there is provided a radially outwardly extending projection 330 on the outside surface of the insert 110. A correspondingly shaped longitudinal groove 430 is provided on the inside surface 203 of the sidewall 202 of the receptacle 210. Also here, there are provided two diametrically oppositely located projections 330 to engage with correspondingly arranged grooves 430.

The fastening element 320 or numerous fastening elements 320 provided on the insert 110 is or are also implemented as a snap element 321. Here, the snap element 321 comprises a radially outwardly extending protrusion 322 to engage with a complementary shaped radial recess 422 provided on the inside surface 203 of the sidewall 202 of the receptacle 210. In the illustration of FIG. 9 the radial recess 422 is illustrated as a through recess. Alternatively, the radial recess 422 has a radial depth that is smaller than the thickness of the sidewall 202. It may be implemented as a blind hole on the inside surface 203 of the sidewall 202.

The radial recess 422 is implemented as and/or provides a counter snap element 421. The radial recess 422 defines or forms a counter fastening element 420. There may be provided numerous pairs of fastening elements 320 and complementary-shaped counter fastening element 420 on the outside of the insert 110 and on the inside surface 203 of the sidewall 202 of the receptacle 210, respectively.

The snap element 321 as illustrated in FIG. 9 may comprise a flexible tongue portion that 124 is flexible radial direction. On the outside surface of the tongue portion 324 there is provided the radially outwardly extending protrusion 322. The tongue portion 324 is confined in circumferential direction by two longitudinally extending slits 326. The slits 326 provide an increased degree of elasticity of the tongue portion 324 and an increased radial flexural capability.

With the presently illustrated examples the insert 110 is provided on the first housing component 100 and the receptacle 210 is provided in the second housing component 200. There are numerous further examples conceivable and within the disclosure of the present application, wherein the insert is provided on the second housing component and wherein the correspondingly-shaped receptacle is provided on the first housing component. Likewise, the specific implementation of radially protruding and radially recessed features, as described in connection with the projection and the groove or in connection with the fastening element and counter fastening element may be interchanged and may be thus provided and implemented in an inverted way compared to the presently shown examples.

### REFERENCE NUMBERS

1 injection device
2 distal direction
3 proximal direction
4 dose incrementing direction
5 dose decrementing direction
6 cartridge
7 bung
8 drive mechanism
9 dose setting mechanism
10 housing
11 trigger
12 dose dial
13 dosage window
14 cartridge holder
15 injection needle
16 inner needle cap
17 outer needle cap
18 protective cap
20 piston rod
21 bearing
22 first thread
23 pressure foot
24 second thread
25 barrel
26 seal
28 threaded socket
30 drive sleeve
31 threaded section
32 flange
33 flange
35 last dose limiter
40 spring
60 clutch
62 insert piece
64 stem
80 number sleeve
81 groove
90 ratchet mechanism
91 ratchet feature
100 housing component
101 connecting end
102 sidewall
103 window
104 longitudinal section
105 outside surface
106 longitudinal section
108 indicator
110 insert
111 sidewall portion
112 end face
114 abutment face
115 flange section
116 gap
120 fastening element
121 snap element
122 recess
130 groove
150 mechanical coding
151 coding feature
152 coding portion
200 housing component
201 connecting end
202 sidewall
203 inside surface
205 outside surface
206 flat section
207 flat section
208 indicator
210 receptacle
211 insert opening
214 end face
218 indicator
220 counter fastening element
221 counter snap element
222 protrusion
230 projection
231 chamfered section
250 mechanical counter coding
251 counter coding feature
252 counter coding portion
320 fastening element
321 snap element
322 protrusion
324 tongue portion
326 slit
330 projection
420 counter fastening element
421 counter snap element
422 recess
430 groove

1.-15. (canceled)

16. A housing of a drug delivery device, the housing comprising:
a first housing component configured to accommodate a cartridge filled with a medicament, the first housing component comprising a first connecting end,
a second housing component configured to accommodate a drive mechanism of the drug delivery device, the second housing component comprising a second connecting end,
an insert provided on one of the first connecting end or the second connecting end,
a receptacle provided on the other one of the first connecting end or the second connecting end, wherein the insert is insertable into the receptacle along a longitudinal direction for mutually fastening the first housing component and the second housing component,
a fastening element provided on the insert,
a counter fastening element having a complementary shape to the fastening element, the counter fastening element provided in the receptacle,
a mechanical coding provided on the insert and comprising a coding feature, wherein the mechanical coding comprises one of a lateral extent of the coding feature or a cross-sectional geometry or shape of the coding feature in a plane transverse to the longitudinal direction,
a mechanical counter coding provided in the receptacle and comprising a counter coding feature, wherein the mechanical counter coding comprises one of a lateral extent of the counter coding feature or a cross-sectional geometry or shape of the counter coding feature in the plane transverse to the longitudinal direction,
wherein the mechanical coding and the mechanical counter coding are operable to prevent an engagement of the fastening element with the counter fastening element when the mechanical coding does not match the mechanical counter coding.

17. The housing according to claim 16, wherein the mechanical coding is integrated into a sidewall portion of the insert.

18. The housing according to claim 17, wherein the mechanical counter coding is integrated into a sidewall of the receptacle.

19. The housing according to claim 17, wherein the first housing component or the second housing component comprises a sidewall, the sidewall comprises a first longitudinal section and a second longitudinal section adjoining the first longitudinal section, wherein the insert is formed by the second longitudinal section and wherein a lateral extent, a cross-sectional geometry, or cross-sectional shape of the insert distinguishes from a respective lateral extent, cross-sectional geometry, or shape of the first longitudinal section of the sidewall.

20. The housing according to claim 16, wherein the mechanical coding is defined by an outer diameter, an outer cross-section, or an outside shape of the insert.

21. The housing according to claim 20, wherein the mechanical counter coding is defined by an inside diameter, an inside cross-section, or an inside shape of the receptacle.

22. The housing according to claim 16, wherein the mechanical counter coding is integrated into a sidewall of the receptacle.

23. The housing according to claim 16, wherein the mechanical counter coding is defined by an inside diameter, an inside cross-section, or an inside shape of the receptacle.

24. The housing according to claim 16, wherein when the mechanical coding does not match the mechanical counter coding, an outer diameter, an outer cross-section, or an outside shape of the insert is larger than a respective inside diameter, inside cross-section or inside shape of the receptacle.

25. The housing according to claim 16, wherein one of the fastening element or the counter fastening element comprises a radial recess to engage with a complementary-shaped radial protrusion of the other one of the fastening element or the counter fastening element.

26. The housing according to claim 25, wherein the fastening element comprises a snap element to engage with a complementary-shaped counter snap element of the counter fastening element.

27. The housing according to claim 25, wherein when the mechanical coding does not match the mechanical counter coding, an outer diameter, an outer cross-section, or an outside shape of the insert is smaller than a respective inside diameter, inside cross-section or inside shape of the receptacle less a radial extent of the radial protrusion.

28. The housing according to claim 16, further comprising:
a groove provided on one of the insert or the receptacle, the groove extending along the longitudinal direction, and
a projection provided on the other one of the insert or the receptacle, the projection configured to slide along the groove upon insertion of the insert into the receptacle to rotationally lock the first housing component relative to the second housing component.

29. The housing according to claim 28, wherein the first housing component or the second housing component comprises a sidewall, the sidewall comprises a first longitudinal section and a second longitudinal section adjoining the first longitudinal section, wherein the insert is formed by the second longitudinal section and wherein a lateral extent, a cross-sectional geometry, or cross-sectional shape of the insert distinguishes from a respective lateral extent, cross-sectional geometry, or shape of the first longitudinal section of the sidewall.

30. The housing according to claim 29, wherein a diameter of the second longitudinal section of the sidewall is larger than a diameter of the first longitudinal section of the sidewall.

31. An injection device for injecting a dose of a medicament and comprising the housing of claim 16, the injection device comprising:
the housing,
the cartridge arranged inside the housing, the cartridge comprising a barrel filled with the medicament and sealed in a proximal longitudinal direction by a movable bung, and
the drive mechanism arranged inside the housing, the drive mechanism comprising a piston rod operable to exert a distally directed dispensing force onto the bung of the cartridge.

32. A kit of parts comprising:
a first housing comprising:
a first housing component configured to accommodate a cartridge filled with a medicament, the first housing component comprising a first connecting end,
a second housing component configured to accommodate a drive mechanism of the drug delivery device, the second housing component comprising a second connecting end,
a first insert provided on one of the first connecting end or the second connecting end,
a first receptacle provided on the other one of the first connecting end or the second connecting end, wherein the first insert is insertable into the first receptacle along a longitudinal direction for mutually fastening the first housing component and the second housing component,
a first fastening element provided on the insert,
a first counter fastening element having a complementary shape to the first fastening element, the first counter fastening element provided in the first receptacle,
a first mechanical coding provided on the first insert and comprising a first coding feature, wherein the first mechanical coding comprises one of a lateral extent of the first coding feature or a cross-sectional geometry or shape of the first coding feature in a plane transverse to the longitudinal direction,
a first mechanical counter coding provided in the first receptacle and comprising a first counter coding feature, wherein the first mechanical counter coding comprises one of a lateral extent of the first counter coding feature or a cross-sectional geometry or shape of the first counter coding feature in the plane transverse to the longitudinal direction,
wherein the first mechanical coding and the first mechanical counter coding are operable to prevent an engagement of the first fastening element with the first counter fastening element when the first mechanical coding does not match the first mechanical counter coding; and
a second housing comprising:
a third housing component configured to accommodate a second cartridge filled with a second medicament, the third housing component comprising a third connecting end,
a fourth housing component configured to accommodate a second drive mechanism of the drug delivery device, the fourth housing component comprising a fourth connecting end,
a second insert provided on one of the third connecting end or the fourth connecting end,
a second receptacle provided on the other one of the third connecting end or the fourth connecting end, wherein the second insert is insertable into the second receptacle along a second longitudinal direction for mutually fastening the third housing component and the fourth housing component,
a second fastening element provided on the second insert,
a second counter fastening element having a complementary shape to the second fastening element, the second counter fastening element provided in the second receptacle,
a second mechanical coding provided on the second insert and comprising a second coding feature, wherein the second mechanical coding comprises one of a lateral extent of the second coding feature or a cross-sectional geometry or shape of the second coding feature in a plane transverse to the second longitudinal direction,
a second mechanical counter coding provided in the receptacle and comprising a second counter coding feature, wherein the second mechanical counter coding comprises one of a lateral extent of the second counter coding feature or a cross-sectional geometry or shape of the second counter coding feature in the plane transverse to the second longitudinal direction,
wherein the second mechanical coding and the second mechanical counter coding being operable to prevent an engagement of the second fastening element with the second counter fastening element when the second mechanical coding does not match the second mechanical counter coding,
wherein the coding feature of the first housing distinguishes from the second coding feature of the second housing with regard to at least one of:
a lateral extent,
a cross-sectional geometry, or
a cross-sectional shape in the plane transverse to the longitudinal direction.

33. The kit according to claim 32, wherein:
at least one of an outer lateral extent, an outer cross-sectional geometry, or an outer shape of the insert of the first housing in the plane transverse to the longitudinal direction is larger than a respective inner lateral extent, inner cross-sectional geometry, or inner shape of the second receptacle of the second housing.

34. The kit according to claim 32, wherein:
the outer lateral extent, the outer cross-sectional geometry, or outer shape of the insert of the first housing in the plane transverse to the longitudinal direction is smaller than a respective inner lateral extent, inner cross-sectional geometry, or inner shape of the second receptacle of the second housing less a radial extent of a radial protrusion of one of the fastening element or counter fastening element.
